(19) United States
(12) Patent Application Publication
(10) Pub. No.: US 2016/0256165 A1
(43) Pub. Date: Sep. 8, 2016

(54) SURGICAL STAPLE REMOVER WITH REMOVABLE FRONT END

(71) Applicant: Sinn RX, LLC, Miami Beach, FL (US)

(72) Inventors: Matthew Sinnreich, Doral, CA (US); Wayne Vassello, Boca Raton, FL (US)

(21) Appl. No.: 14/637,155

(22) Filed: Mar. 3, 2015

(51) Int. Cl. A61B 17/076

(52) U.S. Cl. CPC A61B 17/076

(57) ABSTRACT

A surgical staple remover apparatus is disclosed. The surgical staple remover apparatus includes a handle located at a rear of the apparatus, a housing having an interior volume and a distal opening, and a removable front end located in the distal opening. The front end comprises an upward sloped jaw element comprising a pair of parallel jaws, a movable anvil arm for pressing against a crown of the staple slated for removal, and a movable dual hook arm including a dual hook element. The staple remover further includes a lever mechanically coupled to the anvil arm and the dual hook arm, wherein moving the lever closer to the handle results in the anvil arm and the dual hook arm moving towards the jaw element, thereby deforming the surgical staple and the dual hook arm retracting the deformed surgical staple under the strip element and held in place by same.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The invention disclosed broadly relates to the field of medical devices and, more particularly, relates to the field of devices for automating the process of removing surgical staples.

### BACKGROUND OF THE INVENTION

The use of surgical staples in the medical industry for closing wounds or incisions in the skin of a patient has grown over the last decade due to its advantages over thread sutures. One of the main advantages of surgical staples over thread sutures is the reduced amount of time required for surgical staples to be implanted. In cases where large incisions are made, the use of surgical staples can, for example, reduce the length of time required for the suturing process and thus the length of time the patient must be maintained under anesthesia.

Conventional surgical staples comprise an elongated crown and an L-shaped portion on each end of the crown, wherein when implanted in a patient, the crown is located on the exterior of the skin of the patient and the L-shaped portions are bent in a downward direction so that the ends of the L-shaped portions are opposed, thereby incising and gripping the skin. The aforementioned conventional surgical staple may be removed from the skin of a patient by deforming the staple crown into a U-shaped configuration. This causes the L-shaped legs of the staple to shift upwardly and outwardly so that they may be lifted away from the patient's skin.

A conventional surgical staple remover comprises a scissor-like device having a working end that includes two parallel hooks that are inserted under a surgical staple to be removed. One handle of the remover terminates in an anvil that includes a downward facing footprint situated between the two hooks and wherein the anvil is placed on top of the crown of the surgical staple to be removed. When the conventional surgical staple remover is gripped and contracted by a user, the downward facing footprint of the anvil applies force to the top of the crown of the surgical staple, thereby deforming the staple crown into a U-shaped configuration. Consequently, the L-shaped legs of the staple are moved upwardly and outwardly, thereby lifting away from the patient's skin.

One of the disadvantages of a conventional surgical staple remover is that it does not adequately deal with the final disposition of the surgical staple being removed. It is common to have surgical staples jump into the air or fall away during removal. Personnel must then go about finding and disposing of the removed surgical staple and sterilizing anything the staple came into contact with. It is unsanitary to allow removed surgical staples to come into contact with individuals or things since implanted surgical staples have resided within a human's body and may contain biologically hazardous residue that could contaminate individuals and locations. Further, the process of cleaning up after the conventional removal of surgical staples is time consuming and expensive since proper decontamination and sterilization procedures, employing the use of costly protective equipment and cleaning materials, must be undertaken. Further, during an operation on a patient, it is imperative that all removed staples are accounted for, lest the removed staple falls into an open incision unnoticed.

Another disadvantage of a conventional surgical staple remover is that it requires that each removed surgical staple is immediately disposed of. That is, the doctor or technician must remove a surgical staple, place it in a receptacle, and then return to the wound to remove the next surgical staple. This is problematic as it requires that the doctor or technician temporarily lose sight of the wound while he disposes of the removed surgical staple. A further disadvantage of a conventional surgical staple remover is that the remover must come into contact with the patient's skin, which can be contaminated with biological material that may be infectious. This, in turn, contaminates the surgical staple remover, which must be properly decontaminated and sterilized, thereby causing the expenditure of time and resources.

Therefore, a need exists to overcome the problems with the prior art as discussed above, and particularly for a more effective and efficient surgical staple remover, as well as a more sanitary and easy-to-operate surgical staple remover.

### SUMMARY OF THE INVENTION

Briefly, according to an embodiment, a surgical staple remover apparatus is disclosed. The surgical staple remover apparatus comprises a handle located at a rear of the apparatus, a housing having an interior volume and a distal opening, and a removable front end located in the distal opening. The front end comprises an upward sloped jaw element comprising a pair of parallel jaws, wherein the jaws are configured for placement under a surgical staple slated for removal, a movable anvil arm substantially located within the front end and including a forward facing surface configured for pressing against a crown of the staple slated for removal, thereby deforming the surgical staple, and a movable dual hook arm substantially located within the front end and including a dual hook element configured for grabbing a staple that has been deformed, wherein the anvil arm and the dual hook arm are coupled. The surgical staple remover further includes a lever mechanically coupled to the anvil arm and the dual hook arm, wherein moving the lever closer to the handle results in the anvil arm and the dual hook arm moving towards the jaw element, thereby resulting in the anvil pressing against the crown of the surgical staple, deforming the surgical staple for removal and the dual hook arm grabbing the deformed surgical staple and a strip element located behind the jaw element, such that the dual hook arm retracts the deformed surgical staple under the strip element and held in place by same.

The foregoing and other features and advantages of the embodiments will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings.

FIG. 1 is a perspective view of the surgical staple remover in an open position, in accordance with one embodiment.

FIG. 1A is a perspective view of the surgical staple remover in a disassembled state, in accordance with one embodiment.

FIG. 2 is a perspective view of the surgical staple remover in a closed position, in accordance with one embodiment.

FIG. 3 is a perspective side view of the removable front end of the surgical staple remover in an open position, in accordance with one embodiment.

FIG. 4 is a perspective side view of the removable front end of the surgical staple remover in an interim position, in accordance with one embodiment.

FIG. 5 is a top perspective exploded view of the removable front end of the surgical staple remover, in accordance with one embodiment.

FIG. 5A is a bottom perspective exploded view of the removable front end of the surgical staple remover, in accordance with one embodiment.

FIG. 6 is a cross-sectional view of a deployed surgical staple, in accordance with one embodiment.

FIG. 7 is a top perspective view of the removable front end of the surgical staple remover as it is placed under the surgical staple, in accordance with one embodiment.

FIG. 8 is a side view of the surgical staple remover during use and in an open position, in accordance with one embodiment.

FIG. 9 is a side view of the surgical staple remover during use and in an interim position, in accordance with one embodiment.

FIG. 10 is a side view of the surgical staple remover during use and in another interim position, in accordance with one embodiment.

FIG. 11 is a side view of the surgical staple remover during use and in the fully closed position, in accordance with one embodiment.

FIG. 11A is a perspective view of the surgical staple remover during use and in a retracting position, in accordance with one embodiment.

FIG. 12 is a side view of the surgical staple remover during use and in a retracting position, in accordance with one embodiment.

FIG. 13 is a side view of the surgical staple remover during use and in a fully retracted or open position, in accordance with one embodiment.

FIG. 14 is a side view of the surgical staple remover during use and in a fully retracted or open position, in accordance with one embodiment.

### DETAILED DESCRIPTION

Applicant's surgical staple remover solves problems with the prior art by providing a simple and easy-to-use surgical staple remover that automatically captures deformed and removed surgical staples. The surgical staple remover apparatus improves upon the prior art by definitively dealing with the final disposition of each surgical staple being removed from a patient. The surgical staple remover apparatus eliminates the possibility of having surgical staples jump into the air or fall away during removal. The surgical staple remover apparatus further eliminates the necessity for personnel to find and dispose of the removed surgical staple and sterilize anything the staple came into contact with. The surgical staple remover apparatus eradicates the potential for removed surgical staples to come into contact with, and contaminating, individuals or things. Further, the surgical staple remover apparatus eliminates the need to clean up after the conventional removal of surgical staples, thereby saving time and expense. Also, the surgical staple remover apparatus allows a doctor or technician to undergo the process of removing multiple surgical staples without losing sight of the wound during the process.

Further, the surgical staple remover apparatus provides a surgical staple remover with a minimal number of component parts, thereby reducing the potential for failure or malfunction of the device. Also, the minimal number of component parts allows for quick and inexpensive fabrication of the surgical staple remover, thereby meeting the economic requirements for a disposable surgical staple remover. The surgical staple remover apparatus can be constructed of various metals, as well as plastic. Lastly, the surgical staple remover apparatus provides a removable and replaceable front end—i.e., the portion that touches the removed surgical staples—thereby allowing the front end to be removed after use and replaced. This allows for only a portion of the apparatus to be disposed of after one use, thereby allowing for a majority of the apparatus to be re-used after each use, which reduces waste and related costs.

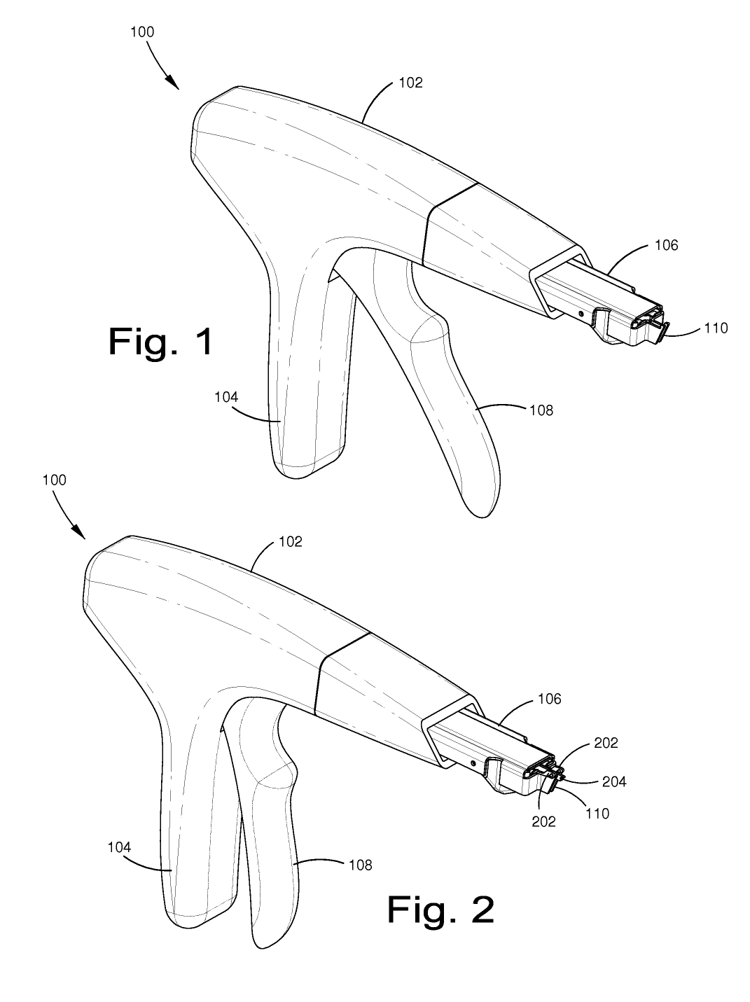

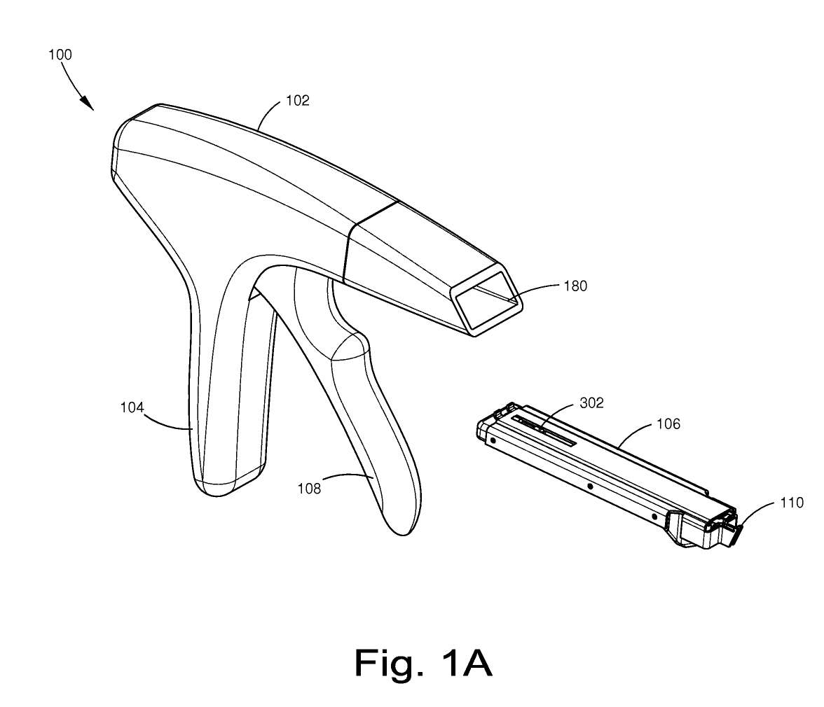

The embodiments of the surgical staple remover apparatus will be described heretofore with reference to FIGS. 1 through 14 below. FIG. 1 is a perspective view of the surgical staple remover 100 in an open position, while FIG. 2 is a perspective view of the surgical staple remover 100 in a fully closed position, in accordance with one embodiment. The apparatus 100 may be composed of a conventional medical device material such as stainless steel and other metal alloys, or a disposable material, such as plastic or a plastic derivative, so that the apparatus (or a portion thereof, such as the removable and replaceable front end) may be disposed after a single use, thereby eliminating the necessity for cleaning or sterilizing the apparatus (or a portion thereof) between uses. One or more of the components that comprise the apparatus 100 may be stamp manufactured from a planar metallic sheet or molded from plastic using conventional plastic molding processes. See FIGS. 5 and 5A which show exploded views of the components of the removable front end 106 of the surgical staple remover 100. The low number of parts, especially moving parts, and the simplicity of the design results in a surgical staple remover 100 that is straightforward and inexpensive to fabricate, thereby meeting the requirements for a disposable medical device.

The apparatus 100 may include a body or housing 102 that comprises a handle 104 (for accommodating the palm of a user's hand) and a protruding element that includes the working end 106 (i.e., a removable front end) of the surgical staple remover, for removing and capturing staples, such as staple 602 (see FIG. 6). The removable front end includes a dual jaw element 110, or a dual-pronged jaw element, that includes a protruding horizontal portion that ends in a vertical portion that extends upwards. The dual jaw element 110 is configured for placement under a surgical staple during removal of said staple. FIG. 1A is a perspective view of the surgical staple remover 100 showing the removable front end 106 removed from the body of housing 102.

The apparatus 100 may also include a lever 108 (for accommodating one or more of the user's fingers), or trigger, that rotates about a pivot point when pressed such that the lever 108 moves closer to the handle 104, i.e., towards the rear of the apparatus 100 or proximally. Note that the angle between the handle 104 and the lever 108 may be substantially in the range of thirty degrees and ninety degrees. In the open position (i.e., at rest), the lever 108 is extended outwards (towards the front end 106) as far as the device allows. In the closed position (FIG. 2), the lever 108 is retracted inwards as far as the device allows.

FIG. 2 shows that the apparatus 100 further includes dual hooks 202 and anvil 204 in the working end 106 of the device. The dual hooks 202 comprise a pair of movable parallel hook elements including J-shaped components that face downwards. The purpose of the dual hooks is to grab, snag or otherwise secure a deformed staple for removal (explained more fully below). Thus, the dual hooks are shaped and configured for grabbing, snagging or otherwise securing a deformed surgical staple. The anvil 204 comprises a movable hard-faced element with a U-shaped end that is configured and shaped for contacting and deforming the crown of a surgical staple (explained more fully below). The anvil 204 is located in between the dual hooks 202 and moves at the same rate as the dual hooks. In one alternative to the dual hooks 202, the disclosed embodiments may include a single hook that performs the same function as the dual hooks.

The operation of the apparatus 100 occurs as follows: the user holds the apparatus 100 via the handle 104, the jaw element 110 of the working end 106 is placed under the staple 602, the lever 108 is pressed by the user's fingers so as to move the lever 108 closer to the handle 104, i.e., proximally, and the working end 106 of the apparatus 100 deforms the staple (via the dual hooks and anvil, as explained more fully below), removes it, and retracts the staple 602 towards the rear of the apparatus 100, or proximally.

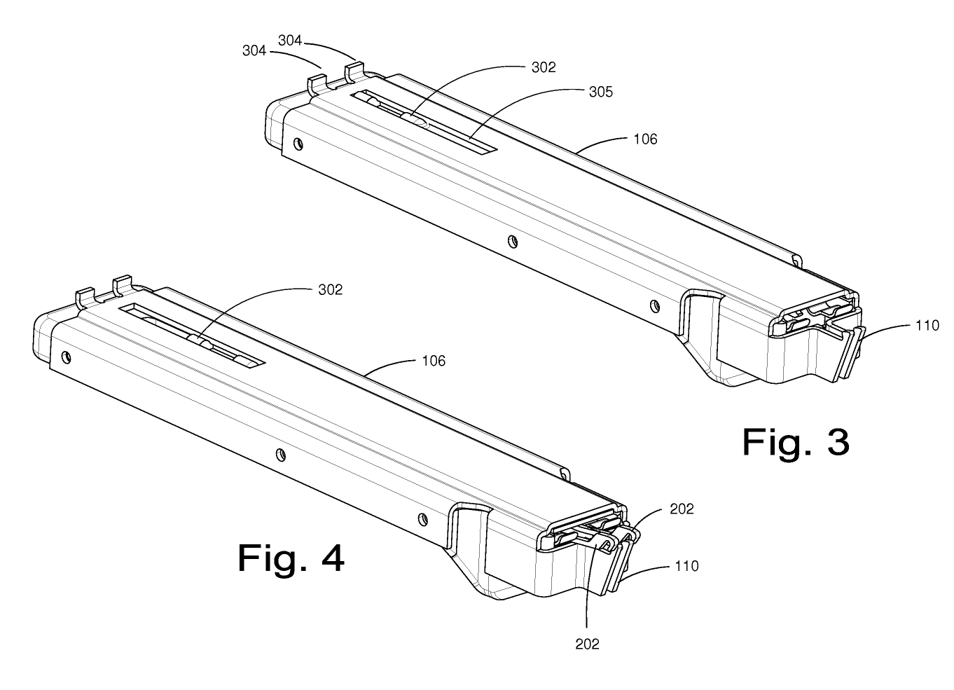

FIG. 3 is a perspective side view of the removable front end 106 of the surgical staple remover 100 in an open position, in accordance with one embodiment. FIG. 3 shows that the removable front end 106 comprises an elongated, substantially rectangular element that may be comprised at least partially out of metal, stainless steel, or the like. The removable front end 106 includes the jaw elements 110 on one end of the substantially rectangular element and two tabs or protrusions 304 located on the other end. The removable front end 106 is configured for being inserted into, and secured to, an orifice 180 in the housing 102. The orifice 180 in the housing 102 is configured to accept, and secure, the removable front end 106 into the housing 102. In one embodiment, the orifice 180 in the housing 102 includes a depression, cavity or opening within the interior of the orifice wherein the depression, cavity or opening is configured to accept the insertion of the two tabs or protrusions 304 located in the removable front end 106, so as to secure the removable front end 106 to the housing 102. In this embodiment, the front end 106 is inserted into the orifice 180 with the tip 110 slightly angled upwards. When the rear of the front end 106 contacts the rear of the orifice 180, the tip 110 is rotated downwards such that the rear of the front end 106 rotates upwards and the tabs or protrusions 304 are inserted into the depression, cavity or opening of orifice 180.

FIG. 3 also shows a sliding tab 302 that is visible through a slot 305 in the top surface of the removable front end 106. The sliding tab 302 is mechanically coupled to the dual hooks 202 and the anvil 204 and movement of the sliding tab 302 results in movement of the dual hooks 202 and the anvil 204. The sliding tab 302 provides a surface configured and shaped for contact with a movable element within the housing 102 (not shown). Note the sliding tab 302 may removably contact the movable element within the housing 102, such that the sliding tab 302 may easily be removed from contacting the movable element within the housing 102 when the front end 106 is removed from the housing 102. In one embodiment, there is no fastener or other securing means between the sliding tab 302 and the movable element within the housing 102, i.e., there is only a surface to surface contact.

As the user holds the apparatus 100 via the handle 104, the lever 108 is pressed by the user's fingers so as to move the lever 108 closer to the handle 104, i.e., proximally, and the movable element within the housing 102 moves outwards or distally, thereby resulting in the sliding tab 302 moving outwards or distally and the dual hooks 202 and the anvil 204 moving outwards or distally. As the user relaxes his grip on the lever 108 and it moves away from the handle 104, the movable element within the housing 102 moves inwards or proximally, thereby resulting in the sliding tab 302 moving inwards or proximally and the dual hooks 202 and the anvil 204 moving inwards or proximally. Since the sliding tab 302 is shown in the retracted or rear position in FIG. 3, the dual hooks 202 and the anvil 204 are also retracted in FIG. 3.

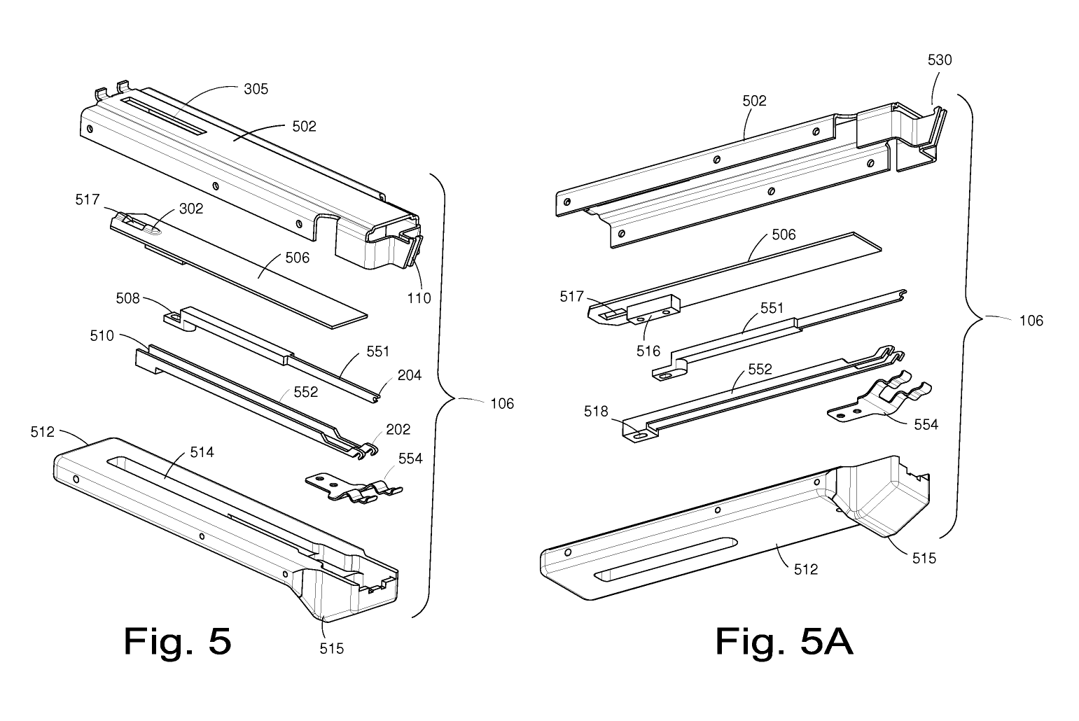

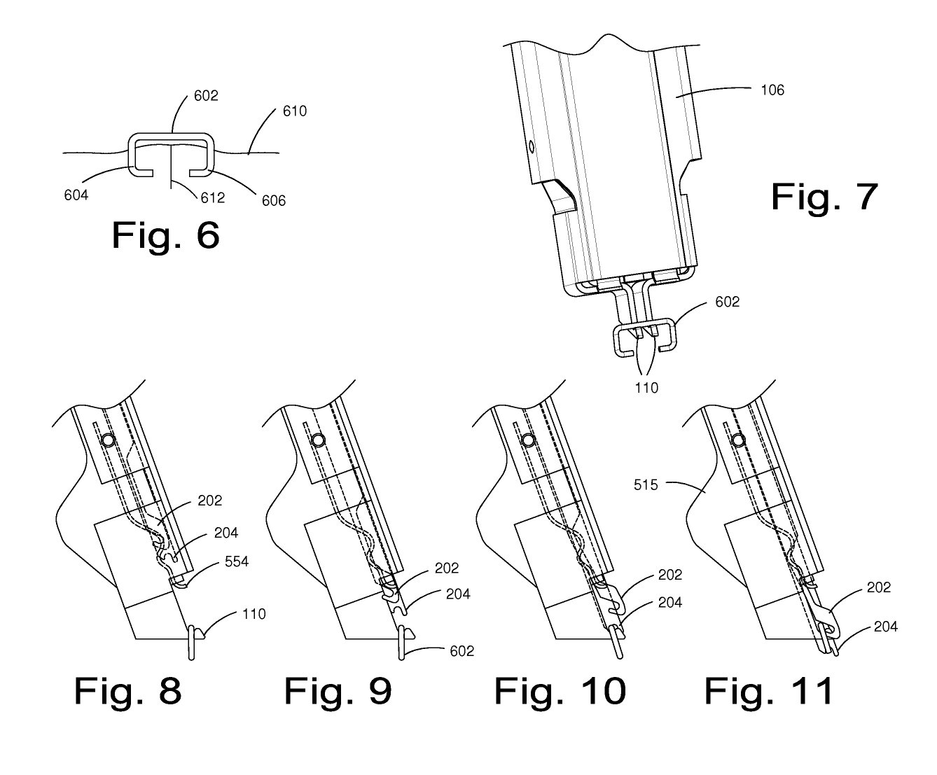

FIG. 4 is a perspective side view of the removable front end 106 of the surgical staple remover 100 in an interim position, in accordance with one embodiment. In FIG. 4 the dual hooks 202 now protrude further from the working end 106 (than in FIG. 3), since it is in an interim position between the open and closed positions. In FIG. 4, since the sliding tab 302 is shown in the deployed or forward position, the dual hooks 202 are also deployed in FIG. 4.

In one embodiment, the removable front end 106 is removable from the orifice 180 in the housing 102 that accepts the front end. In this embodiment, the removable front end 106 may be secured to the housing 102 via a friction fit within orifice 180. A friction fit, or interference fit, is a fastening between two parts which is achieved by friction after the parts are pushed together, rather than by any other means of fastening. A friction fit is generally achieved by shaping the two mating parts so that one or the other, or both, slightly deviate in size from the nominal dimension. One part slightly interferes with the space that the other occupies. The result is that both parts elastically deform slightly to fit together creating an extremely high force which results in extremely high friction between the parts. The friction fit between the removable front end 106 and the orifice in the housing 102 comprises one or more protrusions 304 (see FIG. 3) in the front end 106 that fits into a depression or orifice in the orifice 180 of the housing 102. The friction fit between the removable front end 106 and the orifice 180 in the housing 102 may exhibit a strength index that allows a person to remove the removable front end 106 from the orifice in the housing 102 solely using hand or finger strength.

The removable feature of the front end 106 allows the front end 106 to be removed after a single use (i.e., removing surgical staples) and replaced with another front end 106. Since the front end 106 is the only component of the apparatus 100 that touches a patient, it is most likely the only portion of the apparatus 100 that could be contaminated or infected with communicable afflictions. Thus, this feature does not require that the entire apparatus 100 be discarded after a single use. The removability feature allows the majority of the surgical staple remover 100 to be re-used on other patients while only requiring the front end 106 to be replaced after each use. This feature is advantageous since it leads to less waste, fewer materials being disposed and a cost savings.

FIG. 5 is a top perspective exploded view of the removable front end 106 of the surgical staple remover 100, while FIG. 5A is a bottom perspective exploded view of the removable front end 106 of the surgical staple remover 100, in accordance with one embodiment. The front end 106 includes a top element 502, a sliding element 506, an anvil arm 551, a dual hook arm 552, a strip element 554 and a bottom element 512.

The top element 502 of the front end 106 includes a dual-toothed jaw element 110 protruding from the bottom portion of the front end 106. The dual-toothed jaw element 110 may be integrally formed (such as via a stamping process) from one continuous piece of plastic, metal or alloy (or the like). The dual-toothed jaw element 110 includes two parallel jaws that point in an upward direction. The top surface of the jaws includes a dip or indentation 530 in which a surgical staple is secured while it is deformed during removal. The dual-toothed jaw element 110 may have a size and shape that allows its insertion underneath a crown of a conventional surgical staple 602. The dual-toothed jaw element 110 may further be engineered to allow for slight lateral expansion to ease the deforming of the surgical staple.

The top element 502 includes the slot 305 (which allows access to the sliding tab 302) and includes a box-like shape that houses an interior volume that allows for insertion of the remaining portions of the front end 106, including the elements 506, 551, 552, and 512. The top element 502 also includes the tabs 304, described above.

The sliding element 506 is a substantially flat, planar element that includes the sliding tab 302. The sliding element is located under the top element 502 so as to allow the sliding tab 302 to extend through the slot 305. The sliding element 506 also includes an orifice 517 adjacent to the element 302 and a rectangular shaped protrusion 516 on its underside. The orifice 517 may be shaped and configured to accept the movable element within the housing 102 (described above). Note the orifice 517 may removably contact the movable element within the housing 102, such that the sliding tab 302 may easily be removed from contacting the movable element within the housing 102 when the front end 106 is removed from the housing 102. In one embodiment, there is no fastener or other securing means between the orifice 517 and the movable element within the housing 102, i.e., there is only a surface to surface contact.

The anvil arm 551 is an elongated bar that includes the anvil 204 on its front end. On one end of the anvil arm 551 is an L-shaped feature 508 that includes an orifice 518. The anvil arm 551 is configured and shaped for placement under the sliding element 506. When the anvil arm 551 is placed under the sliding element 506, the L-shaped feature 508 is configured and shaped for mating with the rectangular shaped protrusion 516 on the underside of sliding element 506. That is, the rectangular shaped protrusion 516 may fit securely within the cleft of the L-shaped feature 508. Note also the anvil arm 551 may comprise a thicker body at its proximal end and a thinner body at its distal end.

Dual hook arm 552 comprises two parallel, elongated bars joined on one end, wherein at the other end the dual hooks 202 are located. On one end of the dual hook arm 551 is an L-shaped element 510 that includes an orifice 518. The dual hook arm 551 is configured and shaped for placement under the anvil arm 551. When the dual hook arm 551 is placed under the anvil arm 551, the dual hook arm 551 is configured and shaped for mating with the anvil arm 551. When the dual hook arm 551 is placed under the anvil arm 551, the L-shaped feature 510 is configured and shaped for mating with the rectangular L-shaped feature 508 on the underside of anvil arm 551. That is, L-shaped feature 508 of the anvil arm 551 may fit securely within the cleft of the L-shaped feature 510 of the dual hook arm 551. Note also the dual hook arm 551 may comprise to separate bars (each with a hook at the distal end) wherein the two bars are joined at one end (the proximal end). When the dual hook arm 551 is placed under the anvil arm 551, the dual hook arm 551 is configured and shaped such that the anvil arm 551 may fit in between the two bars of the dual hook arm.

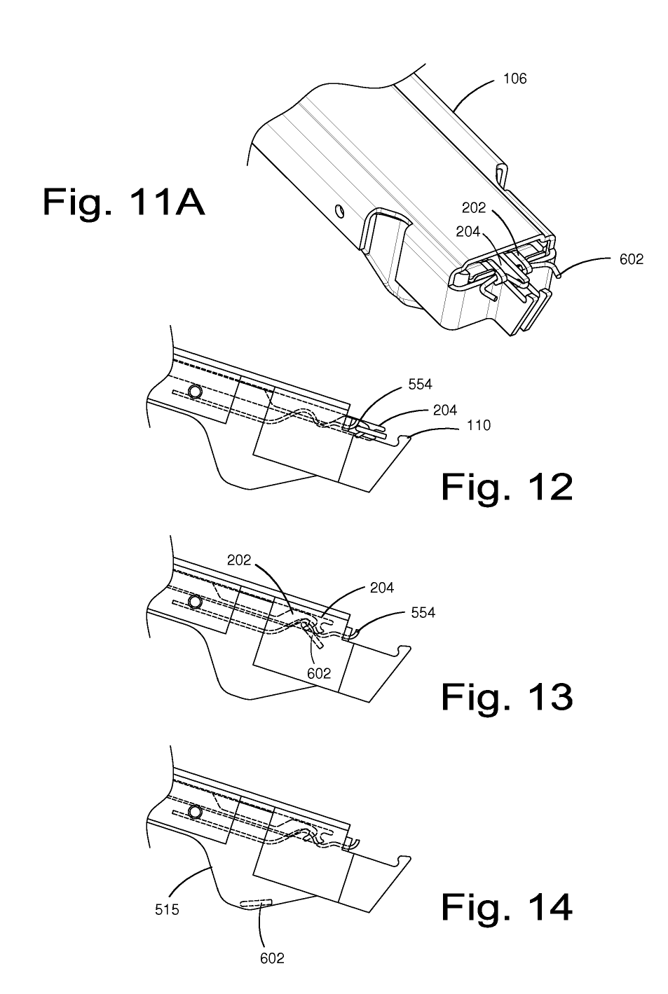

FIG. 5A shows that strip element 554 includes a dual pronged jaw element, which minors the jaw element 110. The strip element 550 is located on top of element 512 (see FIGS. 3 and 11A) such that there is a narrow space between element 512 and strip element 554 to allow the removed and deformed staple to be held securely in the removal and storage process. When the dual hooks 202 and the anvil 204 move a removed surgical staple towards the device 100, the removed surgical staple is moved under the strip element 554 and held in place between the element 512 and strip element 554. The strip element 554 may comprise a strip of plastic or a shape memory alloy that includes one or more bends. The strip element 554 may hold a plurality of removed surgical staples in between the narrow gap between the element 512 and strip element 554. In this manner, multiple surgical staples can be quickly and easily removed, and securely captured by, the apparatus 100. The strip element 554 may include one or more curves or undulations that aid in securing the removed staples and preventing their falling out of the device 100. The strip element 554 may also include one or more orifices for attaching the strip element 554 to the device 100.

FIG. 5A also shows bottom element 512, which includes a channel 514 and a reservoir 515 for housing removed staples. A channel presents a gutter or conduit carved out of a surface to guide movement of another item. In this embodiment, the elements 551 and 552 are located within the channel 514 and said elements move in the path defined by the channel. The purpose of channel 514 is to define the movement of the elements 551 and 552 in response to the movement of the element 506. The element 506 may be mechanically coupled with element 551, which is mechanically coupled with element 552. Also element 554 is mechanically coupled with element 512 and element 512 is mechanically coupled with element 502. Strip element 554 may fit securely within the channel 514 as well. The terms mechanically connected or mechanically coupled refers to a first element being either directly or indirectly attached to a second element using mechanical means, such as via a friction fit, via a fastener or simply the contacting of surfaces.

FIG. 6 is a cross-sectional view of a deployed surgical staple 602, in accordance with one embodiment. The surgical staple 602 is used to close a wound or cut 612 in the skin 610 of a patient. FIG. 6 shows that the surgical staple 602 includes a crown (the horizontal bar running across the top of the staple) and two L-shaped legs 604, 606 on either side of the staple. The L-shaped legs 604, 606 grip the sides of the wound or cut 612 in the skin 610 so as to keep the two sides of the cut or wound together to promote healing and prevent bleeding or exposure to the elements or outside sources of bacteria.

FIG. 7 is a top perspective view of the removable front end 106 of the surgical staple remover 100 as it is placed in under the surgical staple 602, in accordance with one embodiment. FIG. 7 shows that the dual pronged jaws 110 have been placed underneath the staple 602. The skin 610 of the patient is not shown to better illustrate the operation of the surgical staple remover 100. Note the surgical staple remover 100 is still in the fully open position and the lever 108 has not been actuated or pulled back in any manner yet.

FIG. 8 is a side view of the surgical staple remover 100 during use and in a fully open position, in accordance with one embodiment. FIG. 8 shows that the dual pronged jaws 110 have been placed underneath the staple 602. FIG. 8 shows that the dual hooks 202 and the anvil 204 are still in the fully retracted position and do not yet protrude from the working end 106. A tip of the strip 554, however, protrudes from the working end 106.

FIG. 9 is a side view of the surgical staple remover 100 during use and in an interim position, in accordance with one embodiment. In FIG. 9 the surgical staple remover 100 is no longer in the fully open position since the lever 108 has been actuated or pulled back a small distance. As a result, FIG. 9 shows that the dual hooks 202 and the anvil 204 are no longer in the fully retracted position as they now protrude from the working end 106 past the tip of the strip 554. Note, however, that the anvil 204 protrudes past the dual hooks 202, for reasons that will be explained below.

FIG. 10 is a side view of the surgical staple remover 100 during use and in another interim position, in accordance with one embodiment. In FIG. 10 the surgical staple remover 100 is moving closer to the fully closed position since the lever 108 has been actuated or pulled back a greater distance than shown in FIG. 9. As a result, FIG. 10 shows that the dual hooks 202 and the anvil 204 now protrude further from the working end 106 (than in FIG. 9) past the tip of the strip 554. In fact, the anvil 204 protrudes enough to contact the staple 602. In this position, the jaws 110 prevent the staple 602 from moving distally or away from the staple remover 100, and the anvil 204 prevents the staple 602 from moving proximally or towards the staple remover 100. Thus, in this position, the staple 602 is secured between the jaws 110 and the anvil 204. Note that the crown of the staple 602 fits securely within the U-shaped tip of the anvil 204.

FIG. 11 is a side view of the surgical staple remover 100 during use and in the fully closed position, in accordance with one embodiment. In FIG. 11 the surgical staple remover 100 is in the fully closed position since the lever 108 has been actuated or pulled back as far as the device allows. As a result, FIG. 11 shows that the dual hooks 202 and the anvil 204 now protrude further from the working end 106 (than in FIG. 10) past the tip of the strip 554 and even (at least partially) past the jaws 110. In fact, the anvil 204 protrudes enough to deform the staple 602. In this position, the downward facing footprint of the anvil 204 has applied force to the top of the crown of the surgical staple 602, thereby deforming the staple crown into a U-shaped configuration. Consequently, the L-shaped legs of the staple 602 are moved upwardly and outwardly, thereby lifting away from the patient's skin. Once the L-shaped legs 604, 606 have been lifted away from the patient's skin 610, the L-shaped legs no longer grip the sides of the wound or cut 612 in the skin 610. In this state, the staple 602 is now completely out of the patient's skin and ready to be removed from the site.

FIG. 11 further shows that the dual hooks 202 have moved further past the tip of the strip 554 and even (at least partially) past the jaws 110, such that the hooks have moved up and over the staple 602, such that the staple 602 is now located within the J-shaped formation of the dual hooks 202. This position ensures that the staple 602 has now been hooked, snagged or secured by the dual hooks 202 in anticipation of the staple 602 being pulled back by the dual hooks 202 in a later position.

FIG. 11A is a perspective view of the surgical staple remover 100 during use and in a retracting position, in accordance with one embodiment. In FIG. 11A the surgical staple remover 100 is shown after the fully closed position, as the staple remover 100 is returning to the open position, since the user has relaxed pressure on the lever 108 and the lever has moved outwards and towards its original, open position. As a result, FIG. 11A shows that the dual hooks 202 and the anvil 204 have retracted or moved proximally or towards the staple remover 100 and now the dual hooks 202 and the anvil 204 only protrude from the working end 106 past the tip of the strip 554. FIG. 11A also shows how the anvil 204 has deformed the staple 602 in FIG. 11. FIG. 11A shows how the downward facing footprint of the anvil 204 has applied force to the top of the crown of the surgical staple 602, thereby deforming the staple crown into a U-shaped configuration. FIG. 11A also shows how the L-shaped legs of the staple 602 are moved upwardly and outwardly, thereby lifting away from the patient's skin. FIG. 11A further shows that the dual hooks 202 (having earlier hooked, snagged or secured the staple 602) have now pulled back or retracted the deformed staple 602 towards the staple remover 100.

FIG. 12 is a side view of the surgical staple remover 100 during use and in a retracting position, in accordance with one embodiment. In FIG. 12 the surgical staple remover 100 is shown after the fully closed position, as the staple remover 100 is returning to the open position, and as the dual hooks 202 and the anvil 204 are moving back to their original, open position. As a result, FIG. 12 shows that the dual hooks 202 and the anvil 204 have retracted or moved proximally or towards the staple remover 100 and now the dual hooks 202 and the anvil 204 only protrude from the working end 106 past the tip of the strip 554. Also, the staple 602 has been retracted towards the staple remover 100 to the point where the staple 602 has now contacted the strip 554.

FIG. 13 is a side view of the surgical staple remover 100 during use and in a fully retracted or open position, in accordance with one embodiment. In FIG. 13 the surgical staple remover 100 is shown returned to the original, fully open position, since the user has relaxed pressure on the lever 108 and the lever has moved outwards to its original, open position. As a result, FIG. 13 shows that the dual hooks 202 and the anvil 204 have retracted or moved proximally as far as possible into the staple remover 100 and now the dual hooks 202 and the anvil 204 no longer protrude from the working end 106. FIG. 13 also shows how the deformed surgical staple 602 has been moved or pulled by the dual hooks 202 under the strip 554. The curves in the strip 554, as well as the downwards pressure of the strip 544 on the deformed staple 602 prevent the staple from moving outwards or distally form the staple remover 100.

FIG. 14 is a side view of the surgical staple remover 100 during use and in a fully retracted or open position, in accordance with one embodiment. FIG. 14 shows that the surgical staple 602, which was moved or pulled by the dual hooks 202 under the strip 554, has now fallen into the reservoir 515 of the front end 106 of the staple remover 100. Since the staple 602 is now fully disengaged from the dual hooks 202 and the anvil 204, and the surgical staple remover 100 is now in a fully retracted or open position, the surgical staple remover 100 is ready for use in removing another surgical staple from the patient.

In one alternative to the strip 554, a membrane or sphincter (not shown) may be used to strip the deformed and removed surgical staple 602 from the dual hooks 202. In this alternative, the surgical staple 602, is moved or pulled by the dual hooks 202 through, under or behind a membrane or sphincter. In one embodiment, the membrane or sphincter is disposed in a horizontal plane such that the membrane or sphincter is in the same plane as the surface of 506. In one embodiment, the membrane or sphincter is disposed in a vertical plane such that the membrane or sphincter is in a plane perpendicular to the surface of 506. In another embodiment, the membrane or sphincter is disposed in a diagonal plane such that the membrane or sphincter is diagonal to the plane of the surface of 506.

If the staple is moved through said membrane or sphincter, the membrane or sphincter would include an orifice or hole through which the staple is pulled. Once the staple is pulled through, under or behind the membrane or sphincter, said membrane or sphincter provides a barrier to the deformed staple 602 to prevent the staple from moving outwards or distally form the staple remover 100. Subsequently, the staple 602 may then fall into the reservoir 515 of the front end 106 of the staple remover 100. And the surgical staple remover 100 is ready for use in removing another surgical staple from the patient. The membrane or sphincter may be composed of a resilient material such as plastic, rubber or silicone material, for example.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of Applicant's surgical staple remover apparatus.

What is claimed is:

1. A surgical staple remover apparatus, comprising:
a handle located at a rear of the apparatus;
a housing having an interior volume and a distal opening;
a removable front end located in the distal opening, wherein the front end comprises:
an upward sloped jaw element comprising a pair of parallel jaws, wherein the jaws are configured for placement under a surgical staple slated for removal;
a movable anvil arm substantially located within the front end and including a forward facing surface configured for pressing against a crown of the staple slated for removal, thereby deforming the surgical staple; and
a movable dual hook arm substantially located within the front end and including a dual hook element configured for grabbing a staple that has been deformed, wherein the anvil arm and the dual hook arm are coupled;
a lever mechanically coupled to the anvil arm and the dual hook arm, wherein moving the lever closer to the handle results in the anvil arm and the dual hook arm moving towards the jaw element, thereby resulting in the anvil pressing against the crown of the surgical staple, deforming the surgical staple for removal and the dual hook arm grabbing the deformed surgical staple; and
a strip element located behind the jaw element, such that the dual hook arm retracts the deformed surgical staple under the strip element and held in place by same.

2. The surgical staple remover apparatus of claim 1, wherein the forward facing surface of the anvil arm is located in between each hook of the dual hook element of the dual hook arm.

3. The surgical staple remover apparatus of claim 2, wherein the dual hook arm further comprises:
two parallel bars joined at a proximal end and having the dual hook element at the distal end, wherein the anvil arm is coupled to the dual hook arm the proximal end of the dual hook arm.

4. The surgical staple remover apparatus of claim 3, further comprising:
a sliding element mechanically coupled to, and located on top of, the anvil arm and the dual hook arm, wherein the sliding element is further mechanically coupled to the lever.

5. The surgical staple remover apparatus of claim 3, further comprising:
a reservoir located under the strip element, wherein the reservoir includes an interior volume for holding deformed surgical staples that have been retracted by the dual hook arm under the strip element.

6. The surgical staple remover apparatus of claim 5, wherein the front end is secured to the opening of the housing via a friction fit.

7. The surgical staple remover apparatus of claim 6, wherein the friction fit comprises a protrusion in the front end that fits into a depression or orifice in the opening of the housing.

8. The surgical staple remover apparatus of claim 7, wherein the friction fit exhibits a strength index that allows a person to remove the front end solely using hand strength.

9. The surgical staple remover apparatus of claim 1, wherein the apparatus is composed of any one of stainless steel, a metal alloy, plastic, or a plastic derivative.

10. The surgical staple remover apparatus of claim 9, wherein the apparatus is composed of a disposable material.

11. A removable front end for a surgical staple remover apparatus, comprising:
an upward sloped jaw element comprising a pair of parallel jaws, wherein the jaws are configured for placement under a surgical staple slated for removal;
a movable anvil arm substantially located within the front end and including a forward facing surface configured for pressing against a crown of the staple slated for removal, thereby deforming the surgical staple;
a movable dual hook arm substantially located within the front end and including a dual hook element configured for grabbing a staple that has been deformed, wherein the anvil arm and the dual hook arm are coupled and wherein the forward facing surface of the anvil arm is located in between each hook of the dual hook element;
a sliding element mechanically coupled to, and located on top of, the anvil arm and the dual hook arm, wherein moving the sliding element proximally results in the anvil arm and the dual hook arm moving towards the jaw element, thereby resulting in the anvil pressing against the crown of the surgical staple, deforming the surgical staple for removal and the dual hook arm grabbing the deformed surgical staple;
a strip element located behind the jaw element, such that the dual hook arm retracts the deformed surgical staple under the strip element and held in place by same; and
a reservoir located under the strip element and including an interior volume for holding deformed surgical staples that have been retracted by the dual hook arm under the strip element.

12. The removable front end of claim 11, wherein the dual hook arm further comprises:
two parallel bars joined at a proximal end and having the dual hook element at the distal end, wherein the anvil arm is coupled to the dual hook arm the proximal end of the dual hook arm.

13. The removable front end of claim 12, wherein the sliding element includes an orifice on its top surface.

14. The removable front end of claim 13, wherein the anvil arm comprises a U-shaped element on its distal tip.

15. The removable front end of claim 14, wherein the dual hook arm comprises two parallel hook elements on its distal tip.

16. The removable front end of claim 15, wherein the strip element comprises one or more lumps on its surface.

17. The removable front end of claim 11, wherein the front end is composed of any one of stainless steel, a metal alloy, plastic, or a plastic derivative.

18. The removable front end of claim 17, wherein the front end is composed of a disposable material.

19. A surgical staple remover apparatus, comprising:
a handle located at a rear of the apparatus;
a housing having an interior volume and a distal opening;
a removable front end located in the distal opening, wherein the front end comprises:
an upward sloped jaw element comprising a pair of parallel jaws, wherein the jaws are configured for placement under a surgical staple slated for removal;
a movable anvil arm substantially located within the front end and including a forward facing surface configured for pressing against a crown of the staple slated for removal, thereby deforming the surgical staple;
a movable hook arm substantially located within the front end and including a hook element configured for grabbing a staple that has been deformed, wherein the anvil arm and the hook arm are coupled; and
a sliding element mechanically coupled to, and located on top of, the anvil arm and the hook arm;
a lever mechanically coupled to the sliding element, wherein moving the lever closer to the handle results in the anvil arm and the hook arm moving towards the jaw element, thereby resulting in the anvil pressing against the crown of the surgical staple, deforming the surgical staple for removal and the hook arm grabbing the deformed surgical staple;
a membrane located behind the jaw element, such that the hook arm retracts the deformed surgical staple under or behind the membrane and held in place by same; and
a reservoir located under the membrane and including an interior volume for holding deformed surgical staples that have been retracted by the hook arm under the strip element.
